(19) United States
(12) Patent Application Publication
(10) Pub. No.: US 2025/0090021 A1
(43) Pub. Date: Mar. 20, 2025

(54) DETERMINING A STANDOFF DISTANCE TO AN EYE

(71) Applicant: Verily Life Sciences LLC, Dallas, TX (US)

(72) Inventors: Supriyo Sinha, Menlo Park, CA (US); Dimitri Azar, Chicago, IL (US); Hojr Sedaghat Pisheh, Pinole, CA (US); Benjamin Krasnow, Redwood City, CA (US)

(21) Appl. No.: 18/467,587

(22) Filed: Sep. 14, 2023

(51) Int. Cl. A61B 3/14; A61B 3/00; A61B 3/13; A61B 3/16

(52) U.S. Cl. CPC A61B 3/14; A61B 3/0008; A61B 3/0075; A61B 3/13; A61B 3/165

(57) ABSTRACT

Systems, devices, and methods for determining a standoff distance to an eye are provided. In one example, a system may include a structure along a first axis toward an eye, a linear sensor array, and a light source to emit a beam of light toward the linear sensor array along a second axis. The beam of light may comprise a width such that a first portion of the beam of light illuminates a lateral surface of the eye and a second portion of the beam of light passes in front of the eye. The system may determine, based on a measurement of the second portion, a standoff distance between the structure and the eye along the first axis. Another example may include an ultrasonic transducer assembly to emit an ultrasonic pulse toward the eye and detect a reflection of the pulse to determine the standoff distance.

### FIELD

This disclosure relates generally to devices and methods for performing measurements involving eyes, and in particular to devices and methods for determining a standoff distance to an eye. Other aspects are also described.

### BACKGROUND

For many applications, it is important to determine the position or location of the eye relative to a reference point, e.g., a structure or instrument. For example, in air-puff tonometry, the location of the eye relative to the nozzle delivering the air and the optical measurement equipment being utilized may be important for obtaining accurate results. In another example, for retinal imaging, the location of the eye, e.g., to within 1 millimeter (mm), relative to the imaging equipment may be important for obtaining a clear image of the fundus of the eye. In non-ocular diagnostic applications, the location of the eye may be important for augmented reality (AR) and/or virtual reality (VR) applications to accurately determine eye relief relative to the glasses or headset to minimize eye strain and/or maximize optical performance.

### SUMMARY

Implementations of this disclosure include determining a standoff distance to the eye (e.g., between a structure serving as a reference point, such as a nozzle for delivering a jet of air, and an eye). In one example, a system may include a structure disposed along a first axis toward an eye, a linear sensor array disposed distal of the structure, and a light source directed to emit a beam of light toward the linear sensor array along a second axis. The beam of light may comprise a width (e.g., in the first axis) such that a first portion of the beam of light illuminates a lateral surface of the eye and a second portion of the beam of light passes in front of the eye. The system may determine, based on a measurement of the second portion, a standoff distance between the structure and the eye along the first axis. Another example may include an ultrasonic transducer assembly directed to emit an ultrasonic pulse toward the eye and to detect a reflection of the ultrasonic pulse from the eye. The ultrasonic transducer assembly may be configured to emit the ultrasonic pulse and detect the reflection in a field of view that includes the eye and excludes a nose and a forehead near the eye. The system may measure a time of flight based on a difference between a first time corresponding to an emission of the ultrasonic pulse and a second time corresponding to a detection of the reflection. The system may then determine, based on the time of flight, a standoff distance between the structure and the eye along the first axis. Other aspects are also described and claimed.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 a perspective view of an eye instrument, according to at least one embodiment of the present disclosure.

FIG. 2 is a diagrammatic view of an eye instrument having an array-based optical measurement subsystem, according to at least one embodiment of the present disclosure.

FIG. 3 is a top elevation view of an eye instrument having an array-based optical measurement subsystem, according to at least one embodiment of the present disclosure.

FIG. 4 is a front elevation view of an eye instrument having a nozzle, proximity sensor, and an array-based optical measurement subsystem, according to at least one embodiment of the present disclosure.

FIG. 5 is a diagrammatic view of an eye instrument having an array-based optical measurement subsystem, according to at least one embodiment of the present disclosure.

FIG. 6 is a diagrammatic view of an eye instrument having an array-based optical measurement subsystem, according to at least one embodiment of the present disclosure.

FIG. 7 is a diagrammatic view of an eye instrument having an array-based optical measurement subsystem, according to at least one embodiment of the present disclosure.

FIG. 8 is a front elevation view of an eye instrument having a nozzle, proximity sensor, and an array-based optical measurement subsystem, according to at least one embodiment of the present disclosure.

FIG. 9 is a graph depicting a displacement-vs.-time relationship of a cornea deflecting in response to a jet of air from an eye instrument in accordance with at least one embodiment of the present disclosure.

FIG. 10 is a diagrammatic view of an eye instrument utilizing an ultrasonic measurement subsystem, according to at least one embodiment of the present disclosure.

FIG. 11 is a perspective view of ultrasonic optical measurement subsystem of an eye instrument, according to at least one embodiment of the present disclosure.

FIG. 12 is a front elevation view of an ultrasonic measurement subsystem, according to at least one embodiment of the present disclosure.

### DETAILED DESCRIPTION

A standoff distance, or eye standoff, or simply standoff, generally refers to a distance between an eye and a reference point. The standoff distance may be a useful measurement for determining the location of an eye. For example, for ocular diagnostics, the standoff distance may be determined by using a chinrest and a head fixation apparatus to minimize head movement of a patient. Then, an operator looking through an eyepiece can determine the standoff distance with a camera or other optical imaging equipment. If the standoff distance needs to be controlled, the operator can adjust the spacing via a joystick or other device. However, this approach may involve skill of the operator and time to perform the measurements and/or adjustments. For example, determining and/or adjusting the standoff distance may be based on the performance of the operator. Further, this approach may involve implementing large optics which may be cumbersome to deploy. Additionally, this approach may involve inconvenience to the patient when utilizing the head fixation equipment.

In some cases, a system can fix the standoff distance to a repeatable value based on a mechanical design of the equipment or instrument. For example, AR and/or VR glasses, when worn by a user, can fix the standoff distance to a repeatable value. However, this approach might not allow for resolution of the standoff distance greater than a few millimeters. Additionally, during exercise or other activity, the glasses or headset might slip due to motion and/or sweat of the user.

In cases in which cameras are used to determine the standoff distance, the system may require sophisticated algorithms and hardware to obtain the standoff distance in real time. However, frame rates on non-scientific grade imaging sensors are typically limited to 100 frames per second (FPS) at a given region of interest, which may not be sufficient to achieve the necessary temporal resolution. Additionally, the latency associated with the signal acquisition and processing can cause delay in the system.

Implementations of this disclosure address problems such as these by utilizing a linear sensor array or an ultrasonic transducer assembly to determine a standoff distance (e.g., eye standoff, or simply standoff) to an eye. For example, the standoff distance could be measured between a structure serving as a reference point, such as a nozzle for delivering a jet of air, and the eye. Some implementations may include a structure disposed along a first axis toward an eye, a linear sensor array disposed distal of the structure, and a light source directed to emit a beam of light toward the linear sensor array along a second axis. The beam of light may comprise a width (e.g., in the first axis) such that a first portion of the beam of light illuminates a lateral surface of the eye and a second portion of the beam of light passes in front of the eye. A processor executing instructions stored in memory can determine, based on a measurement of the second portion, a standoff distance between the structure and the eye along the first axis. Another example may include an ultrasonic transducer assembly directed to emit an ultrasonic pulse toward the eye and to detect a reflection of the ultrasonic pulse from the eye. The ultrasonic transducer assembly may be configured to emit the ultrasonic pulse and detect the reflection in a field of view that includes the eye and excludes a nose and a forehead near the eye. The processor executing instructions stored in memory can measure a time of flight based on a difference between a first time corresponding to an emission of the ultrasonic pulse and a second time corresponding to a detection of the reflection. The processor can then determine, based on the time of flight, a standoff distance between the structure and the eye along the first axis.

In some implementations, the system described herein can enable an automated measurement of eye standoff (e.g., standoff distance) without involving an operator. The system can utilize a line sensor to measure the profile of a laser beam. In some implementations, the laser may be near infrared where the user would not be able to perceive the illumination, but at a wavelength where silicon sensors are relatively inexpensive and/or widely available (e.g., 700 nanometers (nm) to 1000 nm). In some implementations, the line sensor can read out at greater than 1000 FPS while also being relatively inexpensive and/or widely available. In some implementations, only a single line profile is analyzed, simplifying software from having to analyze two dimensional images.

In some implementations, a system can emit a collimated beam incident on a lens that expands the beam in a beam fan in one dimension. Depending on the position of the eye in the beam fan, the position of the eye can be determined. For example, the system could use a cylindrical lens or a Powell lens. The beam may be collimated in a plane to approximate the same height as an image pixel on the line sensor. The eye might initially be outside of the beam, and as a result, outside of the measurement range. The eye might then move inside of the beam, and as a result, inside of the measurement range to enable a measurement of the standoff distance. For example, the line sensor in the setup may enable determining the eye location by looking at a profile of power on the sensor.

In some implementations, the system may include two cylindrical lenses arranged at either side of the eye at an appropriate angle. In some implementations, the system may emit a beam in a collimated line, e.g., instead of in a beam fan. In some implementations, the system may emit that is collimated to a large size or diameter in multiple dimensions (e.g., at least 10-20 mm in each dimension). In some implementations, an additional optic in the form of a rod lens may be used to focus the light in one dimension on the line sensor. Such implementations can advantageously give insensitivity to misalignment in the multiple dimensions. In some implementations, the system may emit a beam that is collimated to a large size or diameter only in one dimension with the thin dimension being parallel with the linear array. This can advantageously eliminate the need for the rod lens while accommodating misalignment in the axis parallel to the beam propagation but not to the other lateral axis.

In some implementations, the system may utilize a laser diode. In some implementations, the system may utilize a light emitting diode (LED) instead of the laser diode. In some cases, the angle of the beam could be modified, e.g., the normal of the corneal surface might not be perpendicular to the optic axis, to fit the mechanical needs of the instrument or the ergonomic needs of the subject or patient. Since the speed of acquisition in the system may be high, the system can inform an operator whether acquired data is valid or invalid. For example, in fundoscopy, if the user's eye has moved outside of the eye box when the frame was captured, the measurement could be discarded. The system described herein can advantageously avoid this situation. Also, in some cases, the system can be used to calibrate the standoff distance to obtain better resolution. For example, in air puff tonometry, determining the eye's position during the data acquisition can enable the operator to calibrate points of an acquisition curve.

In some implementations, the system may utilize a pair of ultrasound transducers (e.g., one as a receiver and one as a transmitter). The system uses time of flight to determine the position of the eye. A pulse is sent out by the transmitter. A portion of the pulse is returned by the eye through reflection. Other reflections corresponding to echoes can be filtered out and ignored. By measuring the difference in time between the transmitted and received pulse, the distance between the eye and the transmitter can be determined.

In some implementations, a field of view of an ultrasound sensor can be narrow to avoid measuring the forehead or nose when measuring distance to the eye. Transducers of the system can operate at frequencies of 100s of kHz and based on a short distance between the eye and the device (e.g., less than two inches), the refresh rate can be high without undesirable aliasing.

In some implementations, the transducers may be side by side. In some implementations, based on a minimum distance to capture a reflected signal at a receiver, the transducers may point inward. In some implementations, the receiver and the transmitter may be concentric relative to one another. In some implementations, the system may include focused transducer arrays based on sensitivity and/or range requirements. In some implementations, time-of-flight measurements may be made with a single transducer with appropriate electrical switching between transmit and receive modes. In some implementations, to make the transducer ring down as quickly as possible, a backside of the transducer can be covered with an absorptive material such as epoxy impregnated with tungsten powder. This can cause the transmit pulse to be short so that it has decayed before an echo arrives from a nearby object. In some implementations, two transducers may be used which resonate at 400 kHz, and which are designed to be impedance-matched to air.

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

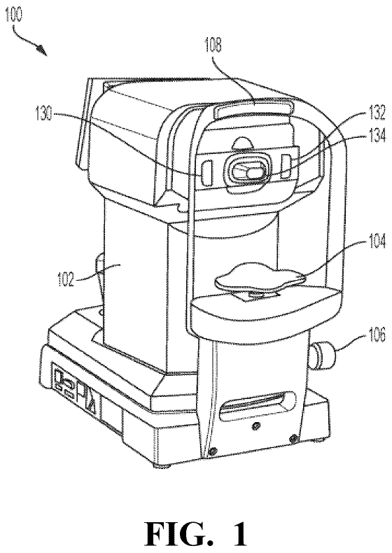

FIG. 1 is a perspective view of an eye instrument, e.g., a non-contact tonometer 100. The tonometer 100 is one example of an instrument that may be configured to determine a standoff distance to an eye. In some cases, the standoff distance may be a distance to a structure (e.g., a reference point) that is along an axis that is normal to the eye (e.g., perpendicular, or orthogonal to the eye). In other cases, the standoff distance may be a distance to the structure that is along an axis at a non-normal angle to the eye (e.g., not perpendicular). The tonometer 100 includes a housing 102 enclosing and/or supporting a plurality of components. The tonometer 100 further includes a chin rest 104, an adjustment dial 106, and a head rest 108. The chin rest 104 and head rest 108 provide stopping features for more consistent and repeatable placement of the user's eye near an eye cup 134. The adjustment dial 106 may be configured to make adjustments in one or more axes to position the user's eye within an acceptable range of positions near the eye cup 134. In some aspects, the adjustment dial 106 may also be used to switch from a left eye position to a right eye position, and vice versa.

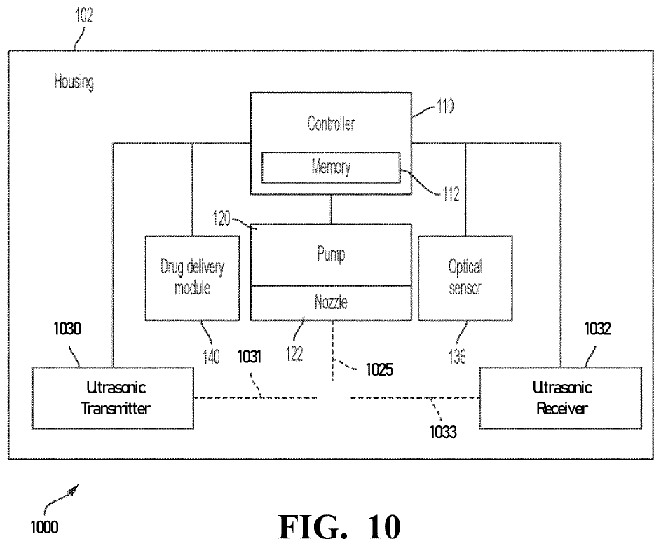

The tonometer 100 further includes a light source 130 and a sensor array 132 to determine the standoff distance. In some embodiments, the tonometer 100 may include an ultrasonic transducer assembly to determine the standoff distance, including as described in FIG. 10. The light source 130 is positioned and oriented to project a beam of light across the eye in a direction transverse to the optical axis of the eye. Similarly, the sensor array 132 is positioned to collect at least a portion of the light from the light source 130, where the collected light travels transversely to the optical axis of the eye. In some aspects, the sensor array 132 is configured to receive the portion of the light that is not obscured by the user's eye/cornea (e.g., the portion of the light that passes in front of the user's eye/cornea). When the eye is moved into the beam of light, e.g., relative to the eye cup 134, a lesser amount of the light from the light source 130 may reach the sensor array 132. Further, when the eye is moved out of the beam of light, a greater amount of the light from the light source 130 may reach the sensor array 132. Additionally, when a jet or puff of air is delivered through the eye cup 134, the cornea may flatten or compress. Accordingly, a greater amount of the light from the light source 130 may reach the sensor array 132 in the moments in time when the cornea is deflected or compressed to let more light pass through. In some aspects, the sensor array includes a plurality of sensor elements positioned along the z-axis or optical axis. The z-axis may be described as being parallel to the direction in which the air puff is delivered, and/or parallel to the optical axis of the eye, e.g., normal to the eye. Accordingly, the number of sensors registering at least a threshold amount of light in the array may correspond to the position of the eye and/or the deflection of the cornea. Thus, the position of the eye, and/or the deflection or compression of the cornea due to the air puff over the period of time in which the eye receives the air puff, may be determined based on the readings from the sensor array 132.

The light source 130 and the sensor array 132 may be controlled by a processor or controller, execute instructions stored in memory, contained within the housing 102. In some cases, the light source 130 and the sensor array 132 may be controlled together to determine a standoff distance between the structure and the eye along the first axis. For example, the standoff distance could be between a structure serving as a reference point, such as the nozzle delivering the air puff, and the eye. The standoff distance may be measured to a resolution of less than 1 mm. In some cases, the light source 130 and the sensor array 132 may be controlled together with the air puff generator components (e.g., pump, valve, etc.) such that the light source 130 and the sensor array 132 are activated in a time period in which the air puff will impinge on the cornea. The light source 130 may be configured to create a collimated beam having a width between 5 mm and 30 mm wide in at least the z-axis. In some embodiments, the collimated beam is between 10 mm and 20 mm wide in the z-axis. In some embodiments, the collimated beam is cylindrical. In other embodiments, the collimated beam is oblong, flat, converging, diverging, or any other suitable profile. In some embodiments, the light source 130 is configured to output a pulsed beam. In other embodiments, the light source 130 is configured to output a continuous beam.

The tonometer 100 may be a benchtop or desktop tonometer device. In some embodiments, the tonometer 100 is configured for home use such that the tonometer 100 can be used by the patient without supervision or assistance by a physician. The tonometer 100 may include one or more user controls for performing an eye position measurement (e.g., standoff distance relative to a structure, such as the nozzle) and/or an intraocular pressure (IOP) measurement (e.g., deflection of the cornea). The tonometer 100 may also include a user interface device for displaying instructions, providing feedback for the user or appropriate positioning of the eye along the air puff axis, displaying the IOP measurements, and/or any other suitable interface function. Tonometers configured for home use may be desirable and are advantageous in some aspects. For example, accurate, frequent, and more immediate IOP measurements may allow for a more tailored or individual treatment regimen. In this regard, if the patient obtains more measurements throughout the day at various times, the patient may obtain more information about the patient's variations in IOP throughout the day. Accordingly, the physician and the patient may create a treatment schedule or regimen that more precisely fits the patient's indications. In other embodiments, the tonometer 100 may be configured for use by a physician, for example, in an ophthalmologist's or optometrist's office. In other embodiments, the tonometer 100 may be a mobile or portable device. For example, the tonometer 100 may be configured for handheld operation. For example, the tonometer 100 may include a rechargeable battery so that the tonometer 100 can be used without being plugged in to a power outlet.

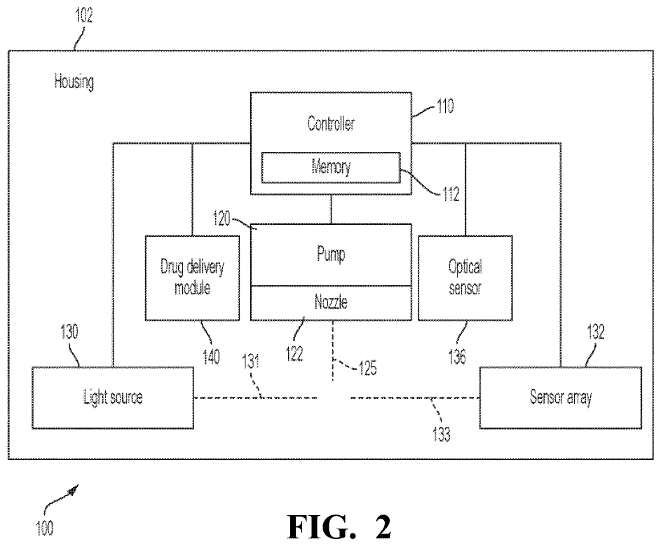

FIG. 2 is a diagrammatic view of an eye instrument (e.g., a tonometer 100). The tonometer 100 shown in FIG. 2 may be similar in some aspects to the tonometer 100 shown in FIG. 1. In this regard, the tonometer 100 shown in FIG. 2 includes a housing 102, a light source 130, and a sensor array 132. The light source 130 is configured to emit a beam of light along a first axis 131. The sensor array 132 is configured to receive light along at least a second axis 133. The tonometer 100 further includes a pump 120 and a nozzle 122 configured to generate an air puff or air jet. The air puff travels along an axis 125. The axis 125 may be aligned with, or substantially aligned with, an optical axis of the eye. Further, the axis 125 may be orthogonal, perpendicular, or otherwise transverse to one or both of the first axis 131 and/or the second axis 133. In some cases, the nozzle 122 may comprise a structure that serves as a reference point for determining a standoff distance to the eye. In other cases, another structure may be used (e.g., an optical sensor 136).

The tonometer 100 further includes a drug delivery module 140 configured to deliver or administer a pharmaceutical agent to the patient's eye. For example, the drug delivery module 140 may be configured to produce a stream or mist of an ophthalmic fluid to the eye as part of a treatment regimen. In some aspects, the delivery of the pharmaceutical agent by the module 140 may be based on the IOP measurements obtained by the tonometer 100. The tonometer 100 further includes an optical sensor 136 different from the sensor array 132. The optical sensor 136 may be configured for proximity measurements, in some embodiments. For example, the optical sensor 136 may be configured to determine whether the eye is within a suitable range of the nozzle 122. In some aspects, the optical sensor 136 may be controlled by the controller 110 for determining whether there is a user at the tonometer to activate the components of the tonometer 100. In some aspects, the optical sensor 136 may be used for blink detection. In this regard, the controller 110 may be configured to detect a blink based on optical measurements from the optical sensor 136, and to activate the pump 120 and/or nozzle 122 after detecting the blink. In some aspects, the pump 120 includes a valve, and the controller 110 may be configured to activate the valve to release the puff of air. In other embodiments, the controller 110 may detect blinks using the light source 130 and the optical sensor array 132. For example, the controller 110 may detect a momentary increase in the corneal profile based on signals from the optical sensor array and determine that a blink has occurred.

The controller 110 can determine, based on a measurement of the beam of light from the light source 130, past the eye, and reaching the sensor array 132, a standoff distance between the structure and the eye along the axis 125. For example, the standoff distance may be a measurement between a structure serving as a reference point (e.g., the nozzle 122) and the eye. In some cases, the measurement can be performed when the eye is within range as determined by the optical sensor 136.

The controller 110 can also determine, based on a measurement of the beam of light from the light source 130, past the eye, and reaching the sensor array 132, a deflection of the eye. For example, the pressure exerted by the air puff on the cornea increases over a brief period of time (in an example, 15 milliseconds), until it is sufficient to cause temporary applanation or flattening of the cornea, and then a brief period of slight concavity. The pressure may then decrease over a period of time (e.g., 15 milliseconds) such that the cornea flattens again before returning to its normal shape. In both moments of applanation, the tonometer 100 can detect the applanation.

In an example, the static pressure of the air puff on the center of the cornea reaches about 30 mmHg (4.0 kPa or 0.04 atmospheres) above ambient pressure, with an accuracy of about ±1 mmHg, or less than ±1 mmHg. For example, the accuracy of the pressure of the air puff created by the pump 120 and the nozzle 122 may be ±0.05 mmHg, ±0.1 mmHg, ±0.5 mmHg, or any other suitable accuracy. Assuming the patient's IOP is somewhere between 5 mmHg and 30 mmHg, the air puff may result in two separate applanation events—one during the rise time and one during the fall time. In some embodiments, the tonometer 100 may further include one or more pressure sensors within the pump 120, the nozzle 122, and/or at any other suitable position to monitor the pressure of the air puff expelled through the nozzle 122. The controller 110 receiving these measurements then has two separate IOP readings that may be reported separately, averaged, or otherwise.

The tonometer 100 includes the controller 110 and a memory 112 in communication with the controller 110. The memory 112 may store instructions executable by the memory 112 for performing one or more of the functions described above. In some embodiments, the memory 112 may further store standoff distances (e.g., eye positions), IOP measurements, patient-related data, and/or any other suitable type of information. In some embodiments, the memory 112 may store treatment-related data for generating treatment alerts or indicators for the patient. For example, the memory 112 may store general and/or patient-specific IOP thresholds for determining whether to output an alert to the user or a network, or to cause the drug delivery module 140 to deliver a pharmaceutical agent to the patient's eye.

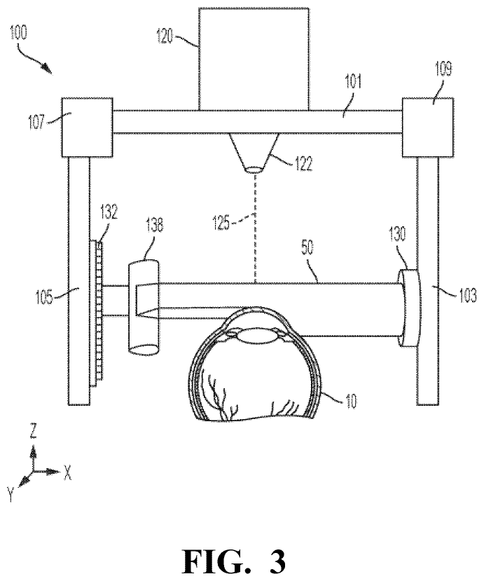

FIG. 3 is a top elevation view of an eye instrument (e.g., the tonometer 100) having a transverse, array-based optical measurement subsystem, according to at least one embodiment of the present disclosure. The tonometer 100 includes a pump 120, a nozzle 122 coupled to a first housing portion 101. The pump 120 and/or the nozzle 122 may comprise a structure serving as a reference point for determining a standoff distance to the eye. The tonometer 100 further includes a light source 130 coupled to a second housing portion 103, and a sensor array 132 coupled to a third housing portion 105. The first housing portion 101 is coupled to the second housing portion 103 by a first connector element 109, and the first housing portion 101 is coupled to the third housing portion 105 by a second connector element 107. In some embodiments, the connector elements 107, 109 are adjustable to adjust the positions of the light source 130, the sensor array 132, and/or the focusing element 138 (e.g., a collimating lens disposed adjacent to the sensor array 132, after the eye 10, such as a rod lens or a cylindrical lens). For example, in some embodiments, the connector elements 107, 109 may include telescoping features, sliding clamps, rack-and-pinion mechanisms, hinges, and/or any other suitable adjustment mechanism.

The pump 120 and nozzle 122 are configured to produce a puff or jet of air along an axis 125. The puff of air may have a controlled profile, pressure, duration, speed, and/or any other pneumatic characteristic. The light source 130 is configured to create a beam 50 of light in a direction that is transverse or perpendicular to the axis 125. It will be understood that the beam 50 may not be exactly perpendicular to the axis 125, in some embodiments. For example, the beam 50 may be centered along an axis or direction that is between 75°−105° with respect to the axis 125. In FIG. 3, the beam 50 is collimated along the x-axis. For example, in some embodiments, the light source 130 may include a laser element. In some embodiments, the laser element may be an infrared (IR) laser element. In other embodiments, the laser element may be a visible light laser element (e.g., configured to emit light in the visible light spectrum). In some embodiments, the light source 130 may include an LED.

In some implementations, the beam 50 comprises a width or thickness in at least the direction of the first axis 125. Accordingly, the beam 50 impinges the eye 10 at a range of depths that includes the cornea. A first portion of the beam 50 is obscured by the eye (e.g., the portion of the light that illuminates a lateral surface of the user's eye/cornea), and a second portion of the beam 50 that is not obscured by the eye 10 (e.g., the portion of the light that passes in front of the user's eye/cornea) continues forward to the focusing element 138. The focusing element 138 may include a rod lens, in some embodiments. In other embodiments, other types of focusing elements may be used, including Fresnel lenses, focusing mirrors, and/or any other suitable type of focusing elements. The focusing element 138 is configured to focus the beam 50 in at least one axis. For example, in FIG. 3, the focusing element 138 is configured to focus the beam 50 in the y-axis (e.g., to a narrow line toward the sensor array 132), but not in the z-axis. In this respect, the focusing element 138 being positioned proximate to the sensor array 132 may allow for some misalignment of the eye in the y-axis. For example, because the beam 50 has a width in the y-axis, there may be a range of suitable positions in the y-axis at which the sensor array 132 can obtain accurate measurements of the standoff distance to the eye from the reference point, the corneal profile, and/or the deflection.

The sensor array 132 includes a plurality of sensor elements arranged along the z-axis (parallel to the axis 125). In the illustrated embodiment, the sensor array 132 is a one dimensional, linear sensor array. Each element of the array 132 may have a width in the z-axis, and a spacing in the z-axis. The width and spacings of the elements of the array 132 may be sufficiently small to detect a position of the eye with great resolution, e.g., less than 1 mm, indicating a standoff distance of the eye to the reference point. The width and spacings of the elements of the array 132 may also be sufficiently small to detect a range of deflections indicating a range of IOPs. For example, each element of the array 132 may have a sensor width ranging between 2 um to 50 um and an inter element spacing ranging between 3 um and 60 um. The elements may also have a height in the y-axis. In some embodiments, the y-axis height of the elements of the array 132 may provide additional flexibility in the eye/corneal position relative to the array 132 and beam 50. The array 132 may include any suitable number of elements, including 20, 48, 64, 128, 256, 512, 1024, 2048 and/or any other suitable number of elements, both greater and smaller. In some embodiments, the elements of the array 132 include photodiodes configured to convert received light into an electrical voltage. The elements of the array 132 may be coupled to one another by a bus. In some embodiments, the array 132 includes a multiplexer to multiplex the signals from each of the electrical elements and transmit the signals to a controller (e.g., controller 110, FIG. 1). In one embodiment, the array 132 includes a Hamamatsu SI 1639-01 (which has 2048 pixels that measure 14 um in width and a total sensor width of 28.672 mm).

In some embodiments, the sensor array 132 may be configured to provide a signal indicating a voltage and/or current for each element of the array 132. In other embodiments, the sensor array 132 may be configured to provide a signal indicating a binary value, for each element, representing whether the sensor element received light above a threshold amount. Accordingly, the signal provided by the array 132 may indicate a number of sensor elements that are illuminated by the light source 130, which is based on the position of the eye in the beam of light, and/or the flattening or deflection of the cornea. Based on the position, the tonometer 100 can determine the standoff distance to the eye (e.g., the distance between the nozzle 122, serving as a reference point, and the eye, along an axis to the eye). Additionally, based on the timing of the flattening of the cornea and the known pressure and characteristics of the air puff, the tonometer 100 can determine the IOP. In this regard, because the beam of light 50 impinges on the eye/cornea from the side, the tonometer 100 may determine the time between the air puff reaching the eye/cornea, and the lateral measurement of the eye/cornea first reaching a minimum.

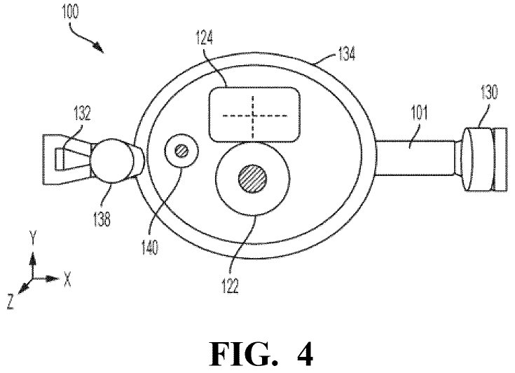

FIG. 4 is a front elevation view of the eye instrument (e.g., the tonometer 100) shown in FIG. 3, according to embodiments of the present disclosure. The tonometer 100 includes the light source 130 positioned opposite and configured to project a beam of light towards the sensor array 132. The focusing element 138 comprises a cylindrical rod lens for focusing beams of light from the light source 130 onto the sensor array 132. The tonometer 100 further includes a housing portion 101, to which the nozzle 122, a drug delivery module 140, and user alignment feature 124 are coupled. The housing portion 101 may comprise an eye cup, in some embodiments. However, the eye cup may be positioned so as not to prevent passage of a beam of light between the light source 130 and the sensor array 132. The drug delivery module 140 may include a dispensing nozzle for producing a stream or mist of a pharmaceutical agent. The user alignment feature 124 may include a display, screen, mirror, and/or any other suitable feature for guiding the user to position the eye/cornea in an acceptable position for performing a standoff distance measurement and/or an IOP measurement. For example, the user alignment feature 124 may include a set of cross hairs, Circles, reticle, or any other suitable type of shape, where the overlapping of the shapes indicates that the user's eye is correctly positioned in at least one of the axes. In some implementations, the nozzle 122 may comprise the reference point for measuring the standoff distance. In some implementations, the user alignment feature 124 may comprise the reference point.

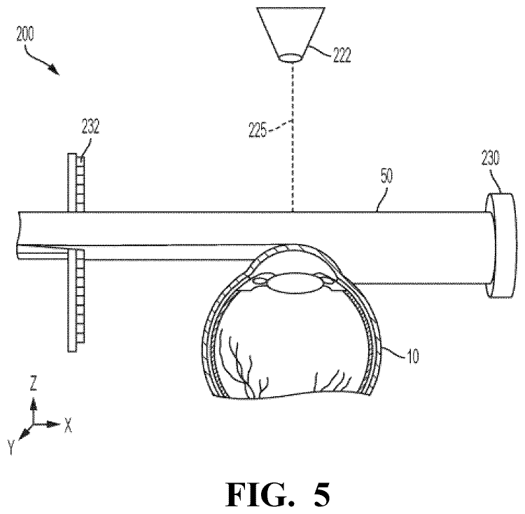

FIG. 5 is a diagrammatic view of an eye instrument (e.g., a tonometer 200) having a transverse, array-based optical measurement subsystem, according to another embodiment of the present disclosure. Similar to the tonometer 100 shown in FIG. 3, the tonometer 200 shown in FIG. 5 includes a nozzle 222, a light source 230, and a sensor array 232. The nozzle 222 may comprise a structure serving as a reference point for determining a standoff distance to the eye. The nozzle 222, light source 230, and sensor array 232 may be coupled to respective housing portions, similar to the embodiment shown in FIG. 3. Further, the positions and orientations of the light source 230 and sensor array 232 may be adjustable by adjustable connection components of the housing, as similarly described above. In the embodiment of FIG. 5, no focusing element is present.

The nozzle 222 may be configured to produce a puff or jet of air along an axis 225. The puff of air may have a controlled profile, pressure, duration, speed, and/or any other pneumatic characteristic. The light source 230 is configured to create a beam 50 of light in a direction that is transverse or perpendicular to the axis 225. It will be understood that the beam 50 may not be exactly perpendicular to the axis 225, in some embodiments. For example, the beam 50 may be centered along an axis or direction that is between 75°-105° with respect to the axis 225. In FIG. 5, the beam 50 is collimated along the x-axis. For example, in some embodiments, the light source 230 may include a laser element. In some embodiments, the laser element may be an infrared (IR) laser element. In other embodiments, the laser element may be a visible light laser element (e.g., configured to emit light in the visible light spectrum). In some embodiments, the light source 230 may include an LED.

In some implementations, the beam 50 comprises a width or thickness in at least the direction of the first axis 225. Accordingly, the beam 50 impinges the eye 10 at a range of depths that includes the cornea. A portion of the beam 50 that is not obscured by the eye 10 (e.g., the portion of the light that passes in front of the user's eye/cornea) continues forward to the sensor array 232. In some aspects, the lack of the focusing element in the embodiment of FIG. 5 may allow for less error or misalignment in eye position than the embodiment shown in FIG. 3, for example.

As similarly described above with respect to FIG. 3, the sensor array 232 includes a plurality of sensor elements arranged along the z-axis (parallel to the axis 225). In the illustrated embodiment, the sensor array 232 is a one-dimensional, linear sensor array. Each element of the array 232 may have a width in the z-axis, and a spacing in the z-axis. The width and spacings of the elements of the array 232 may be sufficiently small to detect a position of the eye with great resolution, e.g., less than 1 mm, indicating a standoff distance of the eye to the reference point. The width and spacings of the elements of the array 232 may also be sufficiently small to detect a range of deflections indicating a range of IOPs. For example, each element of the array 232 may have a sensor width ranging between 2 um and 50 um and an inter-element spacing ranging between 3 um and 60 um. The array 232 may include any suitable number of elements, including 20, 48, 64, 228, 256, 512, 1024, 2048, and/or any other suitable number of elements, both greater and smaller. In some embodiments, the sensor array 232 may include multiple rows of sensor elements stacked in the y-axis. For example, the sensor array 232 may comprise a two dimensional array.

Similar to the tonometer 100, the tonometer 200 may comprise a controller configured to determine, based on signals from the two-dimensional array, a standoff distance to the eye (e.g., the position of the eye/cornea in the y-axis). For example, the standoff distance may be a measurement between a structure serving as a reference point (e.g., the nozzle 222) and the eye. The controller may also be configured to determine, based on signals from the two-dimensional array, and the determined position in the y-axis, the amount of deflection and/or the timing of the deflection of the cornea.

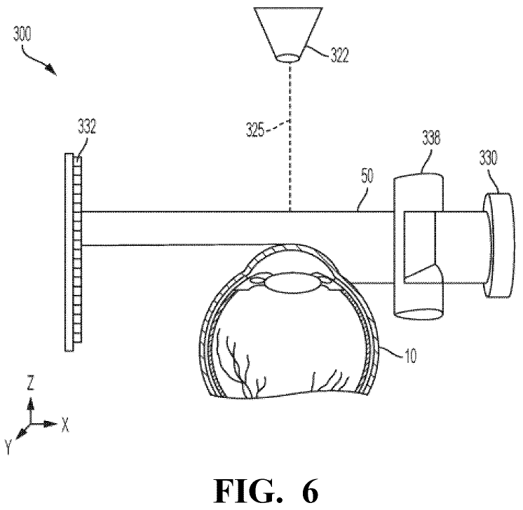

FIG. 6 is a diagrammatic view of an eye instrument (e.g., a tonometer 300) having a transverse, array-based optical measurement subsystem, according to another embodiment of the present disclosure. Similar to the tonometer 100 shown in FIG. 3, the tonometer 300 shown in FIG. 6 includes a nozzle 322, a light source 330, a sensor array 332, and a focusing element 338 (e.g., a collimating lens disposed adjacent to the light source 330, before the eye 10, such as a cylindrical lens or a Powell lens). The nozzle 322 may comprise a structure serving as a reference point for determining a standoff distance to the eye. The nozzle 322, light source 330, sensor array 332, and focusing element 338 may be coupled to respective housing portions, similar to the embodiment shown in FIG. 3. Further, the positions and/or orientations of the light source 330 and sensor array 332 may be adjustable by adjustable connection components of the housing, as similarly described above. In the embodiment of FIG. 6, the focusing element 338 is positioned proximate to the light source 330, instead of proximate to the sensor array 332.

The nozzle 322 may be configured to produce a puff or jet of air along an axis 325. The puff of air may have a controlled profile, pressure, duration, speed, and/or any other pneumatic characteristic. The light source 330 is configured to create a beam 50 of light in a direction that is transverse or perpendicular to the axis 325. It will be understood that the beam 50 may not be exactly perpendicular to the axis 325, in some embodiments. For example, the beam 50 may be centered along an axis or direction that is between 75°-105° with respect to the axis 325. In FIG. 6, the beam 50 is initially collimated along the x-axis, and is then focused to a narrow line by the focusing element 338. The focusing element 338 is positioned proximate to the light source 330. The focusing element 338 may comprise a rod lens, in some embodiments. In other embodiments, the focusing element 338 may comprise a semi-cylindrical lens.

In some implementations, the focused beam 50 comprises a width or thickness in the direction of the first axis 325. Accordingly, the beam 50 impinges the eye 10 at a range of depths that includes the cornea. A portion of the beam 50 that is not obscured by the eye 10 (e.g., the portion of the light that passes in front of the user's eye/cornea) continues forward to the sensor array 332. In some aspects, the beam 50 may have a focal point at or near the sensor array 332. Accordingly, the beam 50 may be converging between the focusing element 338 and the sensor array 332.

As similarly described above with respect to FIG. 3, the sensor array 332 includes a plurality of sensor elements arranged along the z-axis (parallel to the axis 325). In the illustrated embodiment, the sensor array 332 is a one-dimensional, linear sensor array. Each element of the array 332 may have a width in the z-axis, and a spacing in the z-axis. The width and spacings of the elements of the array 332 may be sufficiently small to detect a position of the eye with great resolution, e.g., less than 1 mm, indicating a standoff distance of the eye to the reference point. The width and spacings of the elements of the array 332 may also be sufficiently small to detect a range of deflections indicating a range of IOPs. For example, each element of the array 332 may have a sensor width ranging between 2 um to 50 um and an inter-element spacing ranging between 3 um to 60 um. The array 332 may include any suitable number of elements, including 20, 48, 64, 328, 256, 512, 1024, 2048, and/or any other suitable number of elements, both greater and smaller.

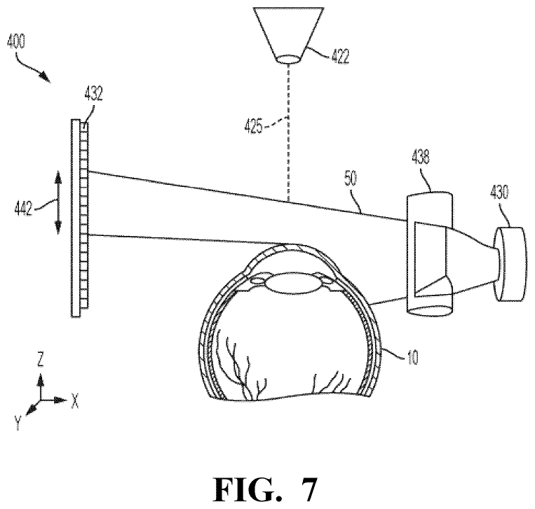

FIG. 7 is a diagrammatic view of an eye instrument (e.g., a tonometer 400) having a transverse, array-based optical measurement subsystem, according to another embodiment of the present disclosure. Similar to the tonometer 100 shown in FIG. 6, the tonometer 400 shown in FIG. 7 includes a nozzle 422, a light source 430, a sensor array 432, and a focusing element 438 (e.g., a collimating lens disposed adjacent to the light source 430, before the eye 10, such as a cylindrical lens or a Powell lens). The nozzle 422 may comprise a structure serving as a reference point for determining a standoff distance to the eye. The nozzle 422, light source 430, sensor array 432, and focusing element 438 may be coupled to respective housing portions, similar to the embodiment shown in FIG. 3. Further, the positions and/or orientations of the light source 430 and sensor array 432 may be adjustable by adjustable connection components of the housing, as similarly described above.

In the embodiment of FIG. 7, the beam 50 produced by the light source 430 is a diverging beam. In some aspects, the light source 430 is configured to emit light as a diverging beam (e.g., a beam fan). In other embodiments, the light source 430 includes one or more optical components (e.g., lens, mirror) configured to create the diverging beam 50 based on light emitted by the light source 430. The tonometer 400 includes a focusing element 438 proximate to the light source 430. The focusing element 438 is configured to focus the diverging beam 50 in the y-axis, creating a fanned beam that diverges in the z-axis and converges in the y-axis. Accordingly, the focused beam 50 comprises a width or thickness in the direction of the first axis 425 that grows as the beam 50 approaches the sensor array 432. The beam 50 impinges the eye 10 at a range of depths that includes the cornea. In some aspects, the diverging nature of the beam 50 may magnify the variation in the amount of light that reaches the sensor array 432 due to the position of the eye and/or the compressed cornea from the air puff. For example, if the cornea compresses 4 mm, the width 442 of the portion of the beam 50 received at the sensor array 432 may change by 4.5 mm. In another embodiment, if the cornea compresses by 4 mm, the width 442 of the portion of the beam 50 that reaches the sensor array 432 may change by 1.2 mm, 1.4 mm, 1.6 mm, 1.8 mm, 2.0 mm, and/or any other suitable amount. The more prominent change in the width 442 based on the diverging beam may improve the resolution of the standoff distance measurements and/or the cornea deflection measurements. For example, the more prominent change in the width 442 may increase the number of sensor elements that receive rays of the beam 50 due to the position of the eye and/or the compression or deflection of the cornea.

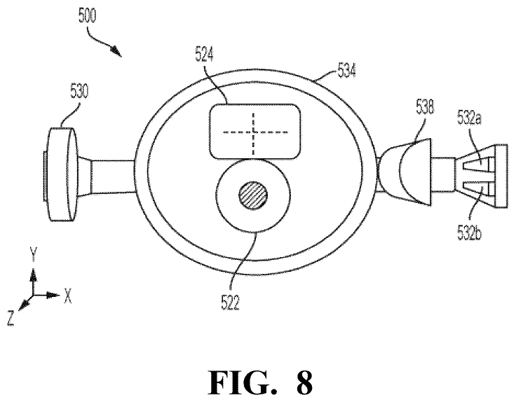

FIG. 8 is a front elevation view of an eye instrument (e.g., a tonometer 500), according to embodiments of the present disclosure. The tonometer 500 includes a light source 530 positioned opposite to, and configured to project a beam of light towards, sensor arrays 532a, 532b. The tonometer 500 includes a focusing element 538 (e.g., a collimating lens disposed adjacent to sensor arrays 532a, 532b, after the eye 10) for focusing beams of light from the light source 530 onto sensor arrays 532a, 532b. The tonometer 500 further includes a housing portion 534, to which a nozzle 522, and a user alignment feature 524 are coupled. The user alignment feature 524 may include a display, screen, mirror, and/or any other suitable feature for guiding the user to position the cornea in an acceptable position for performing an IOP measurement. For example, the user alignment feature 524 may include a set of cross hairs, circles, or any other suitable type of shape, where the overlapping of the shapes indicates that the user's eye is correctly positioned in at least one of the axes. In some implementations, the nozzle 522 may comprise the reference point for measuring the standoff distance. In some implementations, the user alignment feature 524 may comprise the reference point.

The focusing element 538 comprises a semi-cylindrical lens configured to focus light from the light source 530 onto one or both of a first sensor array 532a and/or a second sensor array 532b. The sensor arrays 532a, 532b, may be identical, and arranged in a stacked relationship in the y-axis. In other embodiments, the sensor arrays 532a, 532b may be different sensor arrays. In other embodiments, more than two sensor arrays 532 may be used. For example, the tonometer 500 may include several rows of sensor elements. In some embodiments, a two-dimensional sensor array may be used. Such an arrangement may allow for greater flexibility in the y-axis, since there are multiple rows of sensor elements at different positions in the y-axis to receive the light from the light source 530.

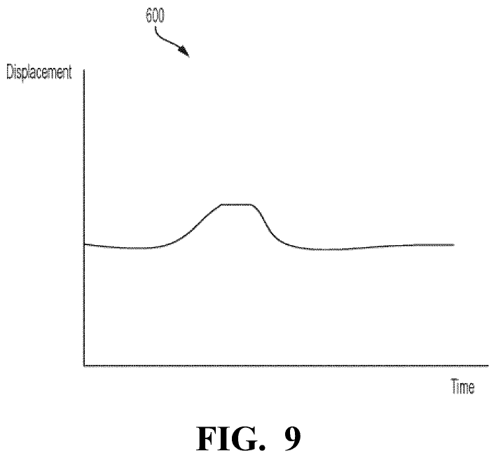

FIG. 9 is a graph of a corneal displacement over time. The corneal displacement is due to the compression of the cornea in response to the air puff from the nozzle of a tonometer. Accordingly, with higher amounts of compression, the graph 600 shows higher amounts of corneal displacement. The displacement reaches a plateau at the point of corneal applanation. At this point, the cornea may become slightly concave. However, because the incident beam approaches the eye laterally, the measured displacement may not detect the momentary concavity of the cornea. The displacement may be measured by a sensor array, such as a linear sensor array. For example, the sensor array may include a plurality of sensor elements corresponding to different depths in the displacement direction. The size and spacing of the sensor elements may be sufficiently small to detect changes in IOP with an accuracy of ±0.05 mmHg, ±0.1 mmHg, ±0.2 mmHg, ±0.5 mmHg, ±1 mmHg, and/or any other suitable accuracy.

In some aspects, a processor (e.g., the controller 110, FIG. 1) is configured to calculate a standoff distance and/or an IOP measurement based on the position of the eye and/or the corneal displacement measurements obtained by the sensor array (e.g., 132, 232, 323, 432, 532a, 532b). In this regard, because the position of the eye and/or the displacement may be associated with the number of sensor elements of the sensor array that are illuminated by the beam of light, the processor may determine the standoff distance and/or the IOP based on the number of sensor elements illuminated by the beam of light. In some aspects, the IOP measurement may be based on known parameters of the air puff, such as the pressure.

The controller could employ any combination of hardware, software, and firmware to perform its functions. The controller could employ a fixed instruction set provided in read-only memory (ROM) or could have an updatable instruction set provided in programmable read-only memory (PROM), electrically erasable programmable read-only memory, flash memory, or any equivalent thereof.

FIG. 10 is a diagrammatic view of an eye instrument (e.g., a tonometer 1000). The tonometer 1000 shown in FIG. 10 may be similar in some aspects to the tonometer 100 shown in FIG. 1 and the tonometer 100 shown in FIG. 2. In this regard, the tonometer 1000 shown in FIG. 10 includes a housing 102 and a controller 110. The tonometer 1000 further includes an ultrasonic transducer assembly comprised of an ultrasonic transmitter 1030 and an ultrasonic receiver 1032. For example, the ultrasonic transducer assembly may be in place of the light source 130 and the sensor array 132. The ultrasonic transmitter 1030 is configured to emit an ultrasonic pulse along a first axis 1031 toward the eye. The ultrasonic receiver 1032 is configured to detect a reflection of the ultrasonic pulse along at least a second axis 1033 from the eye. The tonometer 1000 also includes a pump 120 and a nozzle 122 configured to generate an air puff or air jet. The air puff travels along an axis 125. The axis 125 may be aligned with, or substantially aligned with, an optical axis of the eye. Further, the axis 125 may be orthogonal, perpendicular, or otherwise transverse to one or both of the first axis 131 and/or the second axis 133. In some cases, the nozzle 122 may comprise a structure that serves as a reference point for determining a standoff distance to the eye. In other cases, another structure may be used (e.g., an optical sensor 136).

The tonometer 1000 further includes a drug delivery module 140 configured to deliver or administer a pharmaceutical agent to the patient's eye. For example, the drug delivery module 140 may be configured to produce a stream or mist of an ophthalmic fluid to the eye as part of a treatment regimen. In some aspects, the delivery of the pharmaceutical agent by the module 140 may be based on the IOP measurements obtained by the tonometer 1000. The tonometer 1000 further includes an optical sensor 136 different from the sensor array 132. The optical sensor 136 may be configured for proximity measurements, in some embodiments. For example, the optical sensor 136 may be configured to determine whether the eye is within a suitable range of the nozzle 122. In some aspects, the optical sensor 136 may be controlled by the controller 110 for determining whether there is a user at the tonometer to activate the components of the tonometer 1000. In some aspects, the optical sensor 136 may be used for blink detection. In this regard, the controller 110 may be configured to detect a blink based on optical measurements from the optical sensor 136, and to activate the pump 120 and/or nozzle 122 after detecting the blink. In some aspects, the pump 120 includes a valve, and the controller 110 may be configured to activate the valve to release the puff of air. In other embodiments, the controller 110 may detect blinks using the ultrasonic transducer assembly. For example, the controller 110 may detect a momentary increase in the corneal profile based on signals from the ultrasonic receiver 1032 and determine that a blink has occurred.

The controller 110 can measure a time of flight based on a difference between a first time corresponding to an emission of an ultrasonic pulse from the ultrasonic transmitter 1030 and a second time corresponding to a detection, by the ultrasonic receiver 1032, of a reflection caused by the ultrasonic pulse. The controller 110 can then determine, based on the time of flight, a standoff distance to the eye. For example, the standoff distance may be a measurement between a structure serving as a reference point (e.g., the nozzle 122) and the eye. In some cases, the measurement can be performed when the eye is within range as determined by the optical sensor 136.

The tonometer 1000 includes the controller 110 and a memory 112 in communication with the controller 110. The memory 112 may store instructions executable by the memory 112 for performing one or more of the functions described above. In some embodiments, the memory 112 may further store standoff distances (e.g., eye positions), IOP measurements, patient-related data, and/or any other suitable type of information. In some embodiments, the memory 112 may store treatment-related data for generating treatment alerts or indicators for the patient. For example, the memory 112 may store general and/or patient-specific IOP thresholds for determining whether to output an alert to the user or a network, or to cause the drug delivery module 140 to deliver a pharmaceutical agent to the patient's eye.

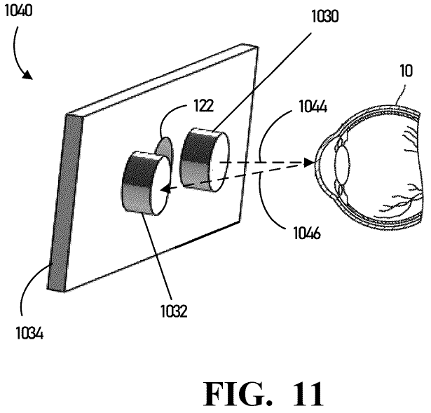

FIG. 11 is a perspective view of an ultrasonic measurement subsystem 1040 of an eye instrument (e.g., the tonometer 1000), according to at least one embodiment of the present disclosure. The subsystem 1040 includes a structure comprising a reference point (e.g., the nozzle 122) and a pair of ultrasound transducers (e.g., one as a receiver and one as a transmitter), such as the ultrasonic transmitter 1030 and the ultrasonic receiver 1032. The subsystem 1100 uses time of flight to determine the position of the eye as described herein.

The transducers of the assembly, e.g., the ultrasonic transmitter 1030 and the ultrasonic receiver 1032, may be side by side. In some implementations, the ultrasonic transmitter 1030 and the ultrasonic receiver 1032 may be angled inward relative to one another along different axes toward the eye (e.g., axes that intersect at the eye). For example, the transducers may each point inward at an angle based on a minimum distance to capture a reflected signal, from the ultrasonic transmitter 1030, at ultrasonic receiver 1032. Angling inward may enable a narrow field of view. In some implementations, the ultrasonic transmitter 1030 and the ultrasonic receiver 1032 may be coupled to the first housing portion 101 shown in FIG. 3. In some embodiments, the connector elements 107, 109 shown in FIG. 3 are adjustable to adjust the positions of the structure, the ultrasonic transmitter 1030, and/or the ultrasonic receiver 1032. For example, in some embodiments, the connector elements 107, 109 may include telescoping features, sliding clamps, rack-and-pinion mechanisms, hinges, and/or any other suitable adjustment mechanism.

The pump 120 and nozzle 122 are configured to produce a puff or jet of air along an axis 125. The puff of air may have a controlled profile, pressure, duration, speed, and/or any other pneumatic characteristic. The ultrasonic transmitter 1030 is configured to generate an ultrasonic pulse 1044 (e.g., frequencies of 100s of kHz) along an axis toward the eye. It will be understood that the ultrasonic pulse 1044 may not be exactly perpendicular to the axis 125, in some embodiments. For example, the ultrasonic pulse 1044 may be centered along an axis or direction that is between 75°-105° with respect to the axis 125. The ultrasonic pulse 1044 impinges the eye 10 and causes a reflection 1046. The ultrasonic receiver 1032 detects the reflection 1046 in a narrow field of view that includes the eye 10 and that excludes other features of the subject near the eye 10, such as a nose and a forehead of the subject. Based on a short distance between the eye and the subsystem 1040 (e.g., less than two inches), the refresh rate from the ultrasonic receiver 1032 can be high without undesirable aliasing. In some implementations, the ultrasonic transmitter 1030 and the ultrasonic receiver 1032 may resonate at 400 kHz and may be designed to be impedance-matched to air.

The ultrasonic transmitter 1030 may be configured to provide a signal indicating an emission of the ultrasonic pulse 1044, and the ultrasonic receiver 1032 may be configured to provide another signal indicating a voltage and/or current corresponding to detection of the reflection 1046. In some embodiments, the ultrasonic receiver 1032 may be configured to provide a signal indicating a binary value representing whether the ultrasonic receiver 1032 received the reflection 1046 above a threshold amount. This may enable the ultrasonic receiver 1032 to distinguish the reflection 1046 from echoes and other noise that can be filtered and ignored. A first signal provided by the ultrasonic transmitter 1030 may indicate a first time corresponding to an emission of the ultrasonic pulse 1044. A second signal provided by the ultrasonic receiver 1032 may indicate a second time corresponding to a detection of the reflection 1046. The controller 110 can measure a time of flight to the eye based on the difference between the first time and the second time. The controller 110 can then determine, based on the time of flight and a known value for the speed of an ultrasonic pulse and its reflection in air, a standoff distance to the eye with great resolution, e.g., less than 1 mm. For example, the standoff distance may represent the distance between the eye and the structure comprising the reference point (e.g., the nozzle 122). Thus, by measuring the difference in time between the transmitted and received pulse on different axes, the distance between the eye and the transmitter can be determined.

In some implementations, the subsystem 1040 may include focused transducer arrays based on sensitivity and/or range requirements (e.g., a plurality of ultrasonic transmitters and ultrasonic receivers). In some implementations, time-of-flight measurements may be made with a single transducer with appropriate electrical switching, by the controller 110, between transmit and receive modes. For example, the controller 110 can control the transducer (e.g., the ultrasonic transmitter 1030) to emit the ultrasonic pulse 1044 at a first time, then switch the transducer to operate as an ultrasonic receiver (e.g., like the ultrasonic receiver 1032) to detect the reflection 1046 at a second time. In some implementations, to make transducer ring-down as quick as possible, a back side of the transducer can be covered with an absorption layer 1034 which may comprise an absorptive material such as epoxy impregnated with tungsten powder. This can cause the transmit pulse to be relatively short so that it decays before an echo arrives from a nearby object.

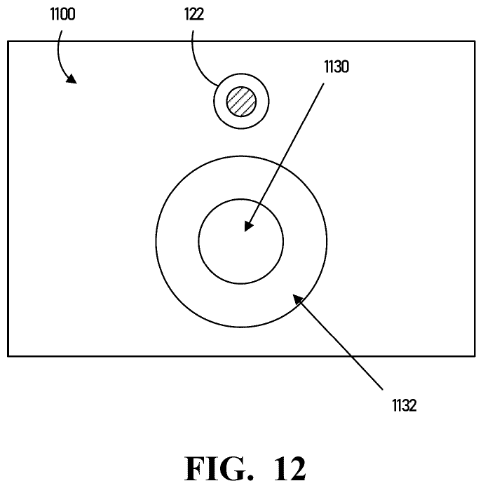

FIG. 12 is a front elevation view of an ultrasonic measurement subsystem 1100, according to at least one embodiment of the present disclosure. Similar to the subsystem 1040 shown in FIG. 11, the subsystem 1100 includes a structure comprising a reference point (e.g., the nozzle 122) for determining a standoff distance to the eye. The subsystem 1040 also includes a pair of ultrasound transducers (e.g., one as a receiver and one as a transmitter), such as the ultrasonic transmitter 1130 and the ultrasonic receiver 1132. The subsystem 1100 uses time of flight to determine the position of the eye as described herein.

The transducers of the assembly, e.g., the ultrasonic transmitter 1130 and the ultrasonic receiver 1132, may be arranged concentrically, e.g., concentric relative to one another, along an axis toward the eye (e.g., an axis that intersects at the eye). In some implementations, the ultrasonic transmitter 1130 and the ultrasonic receiver 1132 may be coupled to the first housing portion 101 shown in FIG. 3. In some embodiments, the connector elements 107, 109 shown in FIG. 3 are adjustable to adjust the positions of the structure, the ultrasonic transmitter 1130, and/or the ultrasonic receiver 1132. For example, in some embodiments, the connector elements 107, 109 may include telescoping features, sliding clamps, rack-and-pinion mechanisms, hinges, and/or any other suitable adjustment mechanism.

As similarly described above with respect to FIG. 11, the ultrasonic transmitter 1130 is configured to generate an ultrasonic pulse (e.g., frequencies of 100s of kHz) along an axis toward the eye. It will be understood that the ultrasonic pulse may not be exactly perpendicular to the axis 125, in some embodiments. For example, the ultrasonic pulse 1044 may be centered along an axis or direction that is between 75°-105° with respect to the axis 125. The ultrasonic pulse impinges the eye 10 and causes a reflection. The ultrasonic receiver 1132, concentric with the ultrasonic transmitter 1130, can detect the reflection 1046 along the same axis in which the ultrasonic transmitter 1130 emitted the pulse. This may enable a narrow field of view that includes the eye 10 and that excludes other features of the subject near the eye 10, such as a nose and a forehead of the subject. Based on a short distance between the eye and the subsystem 1140 (e.g., less than two inches), the refresh rate from the ultrasonic receiver 1032 can be high without undesirable aliasing. In some implementations, the ultrasonic transmitter 1130 and the ultrasonic receiver 1132 may resonate at 400 kHz and may be designed to be impedance-matched to air.

The ultrasonic transmitter 1130 may be configured to provide a signal indicating an emission of the ultrasonic pulse, and the ultrasonic receiver 1132 may be configured to provide another signal indicating a voltage and/or current corresponding to detection of the reflection. In some embodiments, the ultrasonic receiver 1132 may be configured to provide a signal indicating a binary value representing whether the ultrasonic receiver 1132 received the reflection above a threshold amount. This may enable the ultrasonic receiver 1132 to distinguish the reflection from echoes and other noise that can be filtered and ignored. A first signal provided by the ultrasonic transmitter 1130 may indicate a first time corresponding to an emission of the ultrasonic pulse. A second signal provided by the ultrasonic receiver 1132 may indicate a second time corresponding to a detection of the reflection. The controller 110 can measure a time of flight to the eye based on the difference between the first time and the second time. The controller 110 can then determine, based on the time of flight and a known value for the speed of an ultrasonic pulse and its reflection in air, a standoff distance to the eye with great resolution, e.g., less than 1 mm. For example, the standoff distance may represent the distance between the eye and the structure comprising the reference point (e.g., the nozzle 122). Thus, by measuring the difference in time between the transmitted and received pulse on the same axis, the distance between the eye and the transmitter can be determined.

Communication (including but not limited to software updates, firmware updates, or readings from the device) to and from the eye instrument (e.g., the tonometer 100) could be accomplished using any suitable wireless or wired communication technology, such as a cable interface such as a USB, micro USB, Lightning, or Fire Wire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. As explained herein, the disclosed pump devices may be included in a non-contact tonometer, such as in a hand-held non-contact tonometer. Standoff distances and/or IOP measurements may be taken using the tonometer and communicated from the tonometer using the described wireless or wired communication capability.

The logical operations making up the embodiments of the technology described herein may be referred to variously as operations, steps, objects, elements, components, or modules. It should be understood that these may be performed or arranged in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the jet pump for noncontact tonometry. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

The above specification, examples and data provide a complete description of the structure and use of exemplary embodiments of the jet pump for noncontact tonometry as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter. For example, the jet pump could be used to produce controlled puffs of other gases than ambient air, including but not limited to oxygen, nitrogen, helium, and argon, or of gases that contain colorants, odorants, medications, or other materials. Additionally, some or all of the components of the jet pump may be contained within a housing, either alone or with other components such as a battery and/or power supply.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims. Persons skilled in the art will recognize that the devices, systems, and methods described above can be modified in various ways not explicitly described or suggested above. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and, in a manner, consistent with the present disclosure.

Some implementations may include a system, comprising a structure disposed along a first axis toward an eye; a linear sensor array disposed distal of the structure; a light source directed to emit a beam of light toward the linear sensor array along a second axis, wherein the beam of light comprises a width toward the eye such that a first portion of the beam of light illuminates a lateral surface of the eye and a second portion of the beam of light passes in front of the eye; and a processor configured to execute instructions stored in memory to determine, based on a measurement of the second portion of the beam of light via the linear sensor array, a standoff distance between the structure and the eye along the first axis. In some implementations, the structure comprises a pump configured to generate a puff of air and a nozzle in communication with the pump, and the standoff distance is between the nozzle and the eye. In some implementations, the light source comprises a light element and a collimating lens configured to collimate the beam of light along at least one axis. In some implementations, the system may further include a collimating lens coupled to the linear sensor array, wherein the collimating lens is configured to focus the beam of light in a line toward the linear sensor array. In some implementations, the collimating lens is disposed adjacent to the linear sensor array. In some implementations, the system may include a second linear sensor array disposed adjacent to the linear sensor array, wherein the second linear sensor array and the linear sensor array are directed in a third axis toward the light source. In some implementations, the third axis is parallel to the second axis. In some implementations, the processor is further configured to execute instructions stored in memory to receive, from the linear sensor array, a plurality of displacement measurements obtained over a period; and determine, based on the plurality of displacement measurements and the period, an IOP of the eye. In some implementations, the linear sensor array comprises a one dimensional array of photodiodes. In some implementations, the linear sensor array comprises a two dimensional array of photodiodes. In some implementations, the light source is configured to emit light in the visible light spectrum. In some implementations, the light source comprises a light element and a cylindrical lens or a Powell lens configured to expand the beam of light. In some implementations, the system may include a rod lens, or a cylindrical lens configured to focus the beam of light to the linear sensor array.

Some implementations may include a system, comprising a structure disposed along a first axis toward an eye; an ultrasonic transducer assembly directed to emit an ultrasonic pulse toward the eye and to detect a reflection of the ultrasonic pulse from the eye, wherein the ultrasonic transducer assembly is configured to emit the ultrasonic pulse and detect the reflection in a field of view that includes the eye and excludes a nose and a forehead near the eye; and a processor configured to execute instructions stored in memory to measure a time of flight based on a difference between a first time corresponding to an emission of the ultrasonic pulse and a second time corresponding to a detection of the reflection; and determine, based on the time of flight, a standoff distance between the structure and the eye along the first axis. In some implementations, the ultrasonic transducer assembly comprises a transmitter configured to emit the ultrasonic pulse and a receiver configured to detect the reflection. In some implementations, the transmitter and the receiver are angled inward relative to one another along axes toward the eye. In some implementations, the transmitter and the receiver are arranged concentrically relative to one another along a first axis toward an eye. In some implementations, the ultrasonic transducer assembly comprises a transceiver that switches between a transmit mode to emit the ultrasonic pulse and a receive mode to detect the reflection. In some implementations, the system may include an absorption layer arranged on a surface of the ultrasonic transducer assembly, wherein the absorption layer is configured to absorb echoes of the ultrasonic pulse. In some implementations, the ultrasonic transducer assembly includes a transducer configured to resonate at a frequency of at least 400 kHz.

In utilizing the various aspects of the embodiments, it would become apparent to one skilled in the art that combinations or variations of the above embodiments are possible for forming a fan out system in package including multiple redistribution layers. Although the embodiments have been described in language specific to structural features and/or methodological acts, it is to be understood that the appended claims are not necessarily limited to the specific features or acts described. The specific features and acts disclosed are instead to be understood as embodiments of the claims useful for illustration.

What is claimed is:

1. A system, comprising:
a structure disposed along a first axis toward an eye;
a linear sensor array disposed distal of the structure;
a light source directed to emit a beam of light toward the linear sensor array along a second axis, wherein the beam of light comprises a width toward the eye such that a first portion of the beam of light illuminates a lateral surface of the eye and a second portion of the beam of light passes in front of the eye; and
a processor configured to execute instructions stored in memory to:
determine, based on a measurement of the second portion of the beam of light via the linear sensor array, a standoff distance between the structure and the eye along the first axis.

2. The system of claim 1, wherein the structure comprises a pump configured to generate a puff of air and a nozzle in communication with the pump.

3. The system of claim 1, wherein the light source comprises a light element and a collimating lens configured to collimate the beam of light along at least one axis.

4. The system of claim 1, further comprising a collimating lens coupled to the linear sensor array, wherein the collimating lens is configured to focus the beam of light in a line toward the linear sensor array.

5. The system of claim 4, wherein the collimating lens is disposed adjacent to the linear sensor array.

6. The system of claim 1, further comprising a second linear sensor array disposed adjacent to the linear sensor array, wherein the second linear sensor array and the linear sensor array are directed in a third axis toward the light source.

7. The system of claim 6, wherein the third axis is parallel to the second axis.

8. The system of claim 1, wherein the processor is further configured to execute instructions stored in memory to:
receive, from the linear sensor array, a plurality of displacement measurements obtained over a period; and
determine, based on the plurality of displacement measurements and the period, an intraocular pressure (IOP) of the eye.

9. The system of claim 1, wherein the linear sensor array comprises a one dimensional array of photodiodes.

10. The system of claim 1, wherein the linear sensor array comprises a two dimensional array of photodiodes.

11. The system of claim 1, wherein the light source is configured to emit light in the visible light spectrum.

12. The system of claim 1, wherein the light source comprises a light element and a cylindrical lens or a Powell lens configured to expand the beam of light.

13. The system of claim 1, further comprising a rod lens or a cylindrical lens configured to focus the beam of light to the linear sensor array.

14. A system, comprising:
a structure disposed along a first axis toward an eye;
an ultrasonic transducer assembly directed to emit an ultrasonic pulse toward the eye and to detect a reflection of the ultrasonic pulse from the eye, wherein the ultrasonic transducer assembly is configured to emit the ultrasonic pulse and detect the reflection in a field of view that includes the eye and excludes a nose and a forehead near the eye; and
a processor configured to execute instructions stored in memory to:
measure a time of flight based on a difference between a first time corresponding to an emission of the ultrasonic pulse and a second time corresponding to a detection of the reflection; and
determine, based on the time of flight, a standoff distance between the structure and the eye along the first axis.

15. The system of claim 14, wherein the ultrasonic transducer assembly comprises a transmitter configured to emit the ultrasonic pulse and a receiver configured to detect the reflection.

16. The system of claim 15, wherein the transmitter and the receiver are angled inward relative to one another along axes toward the eye.

17. The system of claim 15, wherein the transmitter and the receiver are arranged concentrically relative to one another along a first axis toward an eye.

18. The system of claim 14, wherein the ultrasonic transducer assembly comprises a transceiver that switches between a transmit mode to emit the ultrasonic pulse and a receive mode to detect the reflection.

19. The system of claim 14, further comprising:
an absorption layer arranged on a surface of the ultrasonic transducer assembly, wherein the absorption layer is configured to absorb echoes of the ultrasonic pulse.

20. The system of claim 14, wherein the ultrasonic transducer assembly includes a transducer configured to resonate at a frequency of at least 400 kHz.
